(19) United States
(12) Patent Application Publication
(10) Pub. No.: US 2012/0171716 A1
(43) Pub. Date: Jul. 5, 2012

(54) Measurement of Mitochondrial Membrane Potential to Assess Organ Dysfunction

(75) Inventors: Zhaoli Sun, Perry Hall, MD (US); George Williams, Stuart, FL (US)

(73) Assignee: THE JOHNS HOPKINS UNIVERSITY, Baltimore, MD (US)

(21) Appl. No.: 13/389,861

(22) PCT Filed: Aug. 11, 2010
(86) PCT No.: PCT/US10/45153

§ 371 Date: Mar. 23, 2012

Related U.S. Application Data
(60) Provisional application No. 61/232,982, filed on Aug. 11, 2009.

(51) Int. Cl. C12Q 1/02

(52) U.S. Cl. 435/29

(57) ABSTRACT

The present invention relates to the field of organ transplantation. More specifically, the present invention provides methods for predicting organ function after transplantation. In certain embodiments, the method comprises measuring mitochondrial membrane potential from a biopsy sample from the donor organ. The present invention is also applicable to organ dysfunction in general. More specifically, the methods of the present invention may be useful in formulating prognoses for patients with acute or chronic organ dysfunction due to ischemia, infection, drug injury or age. In this rapid procedure, only small samples of tissue are required, enabling the clinical application of mitochondrial function previously thought impractical.

### CROSS-REFERENCE TO RELATED APPLICATION

This application claims the benefit under 35 U.S.C. §119(e) of U.S. Provisional Application Ser. No. 61/232,982, filed Aug. 11, 2009, which is entirely incorporated herein by reference.

### FIELD OF THE INVENTION

The present invention relates to the field of organ transplantation. The present invention is also applicable to organ dysfunction in general.

### BACKGROUND OF THE INVENTION

Transplantation represents an established procedure in end-stage organ failure patients and routinely produces satisfying, long-term results. However, this surgical therapy is continuously limited by severe and progressive donor organ shortages. Therefore, optimal utilization of all suitable donor organs is mandatory. Current “standard” donor criteria can be significantly liberalized to increase the available donor pool by accepting organs from “marginal donors” making their assessment extremely important. These criteria include advanced age, deterioration of function in the dying donor, and long preservation times. The organ shortage has driven many transplant programs to extend their criteria to accept such donors, but when doing so they are faced with a proportion of kidneys which function poorly or not at all. If physicians could distinguish organs with the potential for good function from organs doomed to failure, the donor pool could be safely expanded. At present, there are no reliable diagnostic methods to measure acute intracellular injury allowing surgeons to accept or discard an organ with questionable function.

### SUMMARY OF THE INVENTION

The present invention relates to the field of organ transplantation. More specifically, the present invention provides methods for predicting organ function after transplantation. In certain embodiments, the method comprises measuring mitochondrial membrane potential (MMP) from a biopsy sample from the donor organ. The present invention is also applicable to organ dysfunction in general. More specifically, the methods of the present invention may be useful in formulating prognoses for patients with acute or chronic organ dysfunction due to ischemia, infection, drug injury or age. In this rapid procedure, only small samples of tissue are required, enabling the clinical application of mitochondrial function previously thought impractical. Indeed, measurement of MMP, by itself or in conjunction with other measurements of mitochondrial function, can be used to predict full, partial or failed organ recovery.

In one embodiment, the present invention provides a method for predicting whether a donor organ is viable for transplantation comprising the steps of isolating mitochondria from a biopsy sample taken from a donor organ; and measuring the MMP of the isolated mitochondria, wherein a MMP level that correlates to MMP levels from cells of a healthy organ indicates that the donor organ is a suitable candidate for transplantation.

In another embodiment, a method for predicting whether an organ will display immediate graft function following transplantation comprises isolating mitochondria from a biopsy sample taken from a donor organ; and measuring the MMP of the isolated mitochondria, wherein a MMP level that correlates to normal mitochondrial function in a healthy cell indicates that the donor organ is likely to exhibit immediate graft function following transplantation.

The organ to be transplanted may comprise any organ including, but not limited to, kidney, liver, heart, pancreas, lung and intestine. In a specific embodiment, the organ is a kidney.

In another embodiment, the present invention provides a method for predicting organ transplant outcome comprising the step of measuring the MMP of mitochondria isolated from a biopsy taken from a donor organ, wherein a MMP level that correlates to altered mitochondrial function or cellular apoptosis is predictive of a negative organ transplant outcome.

In a more specific embodiment, a method for assessing the transplant viability of a donor kidney comprising the steps of isolating mitochondria from a biopsy sample taken from the donor kidney; and measuring the MMP of the mitochondria, wherein a MMP of at least 5000 RFU/μg mitochondria indicates that the donor organ is a viable candidate for transplantation.

Alternatively, a method for predicting whether a donor kidney will display immediate graft function following transplantation may comprise the steps of isolating mitochondria from a biopsy sample taken from the donor kidney; measuring the MMP of the mitochondria, wherein a MMP of at least 5000 RFU/μg mitochondria is indicative that the donor kidney will display immediate graft function following transplantation. The immediate graft function may comprise a serum creatinine level of 3 mg/dl or less at post-operation day 5 (POD5).

The present invention is also applicable to organ dysfunction in general. In certain embodiments, the present invention provides methods for formulating a prognosis of patients with acute or chronic organ dysfunction comprising the steps of isolating mitochondria from a biopsy sample taken from the organ; and measuring the MMP of the isolated mitochondria, wherein a MMP level that correlates to normal mitochondrial function in a healthy organ is predictive of a positive prognosis, and wherein a MMP level that correlates to altered mitochondrial function or cellular apoptosis is predictive of a negative prognosis. In such methods, the organ dysfunction results from ischemia, infection, drug injury, or age.

In further embodiments, the present invention may measure other mitochondrial functions as a way of determining whether an organ is viable for transplantation or assessing organ dysfunction in general. Such assays are known in the art and include, but are not limited to, reactive oxygen species (ROS) production, adenosine (ATP) generation, respiratory chain function, and mitochondrial protein cell sap protein ratios. The present invention may utilize one, some or all of these parameters to screen donor organs for use in transplantation or otherwise assess organ function in general.

In yet another aspect, the present invention provides kits for utilizing the methods described herein. The kits may comprise the equipment, solutions and instructions necessary to obtain a biopsy sample from a donor organ, isolate mitochondria from the sample, measure MMP and/or any other useful parameter in the isolated mitochondria. More specifically, a kit of the present invention may comprise needle biopsy kit components, mitochondria isolation kit components, and/or mitochondrial function assay kit components for measuring MMP, ROS production, ATP generation, mitochondrial respiratory chain function, and/or mitochondrial protein cell sap protein ratios.

### BRIEF DESCRIPTION OF THE FIGURES

FIG. 1 is a graph showing the mitochondrial membrane potential (MMP) for all pre- and post-reperfusion biopsies of donor renal grafts.

FIG. 2 is a graph comparing graft outcome against MMP levels. There was a stepwise increase in MMP that correlated with improved renal function. For purposes of clinical correlation, patients were grouped into three categories with regard to post-operative renal function: (1) Delayed Graft Function (DGF) is defined by the need for dialysis within the first week of transplantation; (2) Non-Immediate Graft Function (NIGF) (also known as Slow Graft Function (SGF)) is defined as a serum creatinine (Cr) level greater than 3 mg/dl at post-operation day 5 (POD5) but no need for dialysis; and (3) Immediate Graft Function (IGF) is defined as a Cr of 3 mg/dl or less at POD5.

FIG. 3 is a graph showing that donor age does not correlate with graft MMP.

FIG. 4 is a graph illustrating that donor age does not correlate with graft function.

FIG. 5 is a graph plotting cold ischemic time (CIT) against serum creatinine on POD5. CIT correlates linearly with post-transplant function.

FIG. 5 is a graph plotting serum creatinine on POD5 against pre-reperfusion MMP. A strong correlation was observed between MMP and post-transplant graft function.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Mitochondrial dysfunction or injury is the hallmark of many pathological processes including cerebral ischemia, neuronal diseases, reperfusion injury and cancer. More specifically, mitochondrial permeability transition (MPT) is the collapse of the electrochemical gradient across the mitochondrial membrane and is recognized as one of the early events of apoptosis. Key factors regulating MPT (Δψm) including calcium, the cellular redox status (including levels of reactive oxygen species) and the mobilization and targeting to mitochondria of Bcl-2 family members.

Mitochondrial membrane potential (MMP) is an important parameter of mitochondrial function and an indicator that the cells will be able to convert oxygen to cellular energy. The measurement of MMP in fresh isolated cells and/or cultured cells is one of the methods used to study signaling mechanisms involved in the initiation of the apoptotic cascade. Loss of MMP is an early event in several types of apoptosis, and can be determined by the MMP-specific fluorescent probe JC-1.

In such studies, the association of low MMP with cellular apoptosis was shown using large samples of cultured or freshly isolated cells. There are no reports of using MMP to predict organ function largely because investigators thought a large amount of tissue was required to separate mitochondria and measure MMP, and the procedures took too long to be clinically useful.

As described herein, the inventors have investigated and optimized methods for the isolation of mitochondria from clinical needle biopsy samples and have demonstrated a co-relation between the level of the MMP and organ function after transplantation. In certain embodiments, the invention comprises a method to measure intact mitochondria function (MMP) from small biopsy samples. The assay can be completed quickly (within one (1) hour) and is suitable for clinical application.

Accordingly, in one aspect, the present invention may be used to assess donor organs prior to transplantation. More specifically, in certain embodiments, the present invention may be used to screen marginal organs for transplantation. In another sense, the present invention may be used to predict organ function after transplantation. Further, the present invention may be used to diagnose delayed graft function and/or primary graft non-function as distinguished from graft rejection thereby diminishing exposure to harmful immunosuppressive drugs.

The methods of the present invention are applicable to all donor organs for transplantation including, but not limited to kidney, liver, heart, pancreas, lung and intestine. The present invention is also applicable in the measurement of tissue injury for all patients with ischemia and/or shock. More specifically, the methods of the present invention are particularly applicable in assessing patients with acute or chronic organ dysfunction due to ischemia, infection, drug injury or age. Indeed, measurement of MMP, by itself or in conjunction with other measurements of mitochondrial function, can be used to predict full, partial or failed organ recovery.

In certain embodiments, a tissue biopsy is obtained from a potential donor organ. Tissue or cell samples can be removed from almost any part of the body. Biopsy methods for obtaining a tissue sample include needle (e.g. fine needle aspiration), endoscopic, and excisional. In particular embodiments, any method for obtaining a minimum of 0.5 mg of tissue is suitable, with the tissue being examined immediately or stored in UW (Univ. of Wisconsin) solution at 4° C. for less than about 12 hours. Variations of these methods and the necessary devices used in such methods are known to those of ordinary skill in the art.

In other embodiments, mitochondria are isolated from the biopsy samples using methods known to those of ordinary skill in the art. The key steps when isolating mitochondria from any tissue or cell are always the same: (i) rupturing of cells by mechanical and/or chemical means and (ii) differential centrifugation at low speed to remove debris and extremely large cellular organelles, followed by centrifugation at a higher speed to isolate mitochondria which are collected. Commercially available kits for isolating mitochondria include, but are not limited to, Mitochondrial Isolation Kit, Catalog No. MITOISO2 (Sigma Aldrich, Inc., St. Louis, Mo.); Mitochondrial Isolation Kit for Tissue, Catalog No. MS850 (MitoSciences, Inc., Eugene, Ore.); Mitochondrial Isolation Kit, Order No. 130-094-532 (Miltenyi Biotech, Inc., Auburn, Cal.); and Mitochondrial Isolation Kit for Tissue, Catalog No. 89801 (Thermo Fisher Scientific, Rockford, Ill.).

Another aspect of the present invention is the measurement of MMP in isolated mitochondria. By way of background, the mitochondrial permeability transition is an important step in the induction of cellular apoptosis. During this process, the electrochemical gradient (referred to as Δ ψ) across the mitochondrial membrane collapses. The collapse is thought to occur through the formation of pores in the mitochondria by dimerized Bax or activated Bid, Bak, or Bad proteins. Activation of these pro-apoptotic proteins is accompanied by the release of cytochrome c into the cytoplasm, which promotes the activation of caspases, which are directly responsible for apoptosis. Basanez et al., 96 PROC. NATL. ACAD. SCI. USA 5492-97 (1999); Desagher et al., 144(5) J. CELL. BIOL. 891-901 (1999); Luo et al., 94 CELL 481-90 (1998); and Narita et al., 95 PROC. NATL. ACAD. SCI. USA 14681-14686 (1998).

Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes. See, e.g., Haugland, 1996 HANDBOOK OF FLUORESCENT PROBES AND RESEARCH CHEMICALS, Sixth Ed., Molecular Probes, Eugene, Ore., pp. 266-274 and 589-594; and Ernster et al., 91 J. CELL BIOL. 227s (1981). For example, by way of illustration and not limitation, the fluorescent probes 2-,4-dimethylaminostyryl-N-methylpyridinium (DASPMI) and tetramethylrhodamine esters (such as, e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential. See, e.g., Murphy et al., 1998 MITOCHONDRIA & FREE RADICALS IN NEURODEGENERATVE DISEASES, Beal, Howell and Bodis-Wollner, Eds., Wiley-Liss, New York, pp. 159-186. Other fluorescent detectable compounds that may be used in the invention include but are not limited to rhodamine 123, rhodamine B hexyl ester, DiOC₆(3), JC-1 [5,5′,6,6′-tetrachloro-1,1′,3,3′-tetraethylbenzimidazolcarbocyanine iodide] (see Reers et al., 260 METH. ENZYMOL. 406 (1995); and Cossarizza, et al., 197 BIOCHEM. BIOPHYS. RES. COMM. 40 (1993)), rhod-2 (see U.S. Pat. No. 5,049,673) and rhodamine 800 (Lambda Physik, GmbH, Gottingen, Germany; see Sakanoue et al., 121 J. BIOCHEM. 29 (1997).

In particular embodiments, the present invention utilizes a unique fluorescent cationic dye, JC-1 (5,5′,6,6′-tetrachloro-1,1′,3,3′-tetraethylbenzimidazolylcarbocyanine iodide), to signal the loss of mitochondrial membrane potential. Smiley et al., 88 PROC. NATL. ACAD. SCI. USA 3671-75 (1991). In healthy non-apoptotic cells, the dye stains the mitochondria bright red. Cossarizza et al., 197(1) BIOCHEM. BIOPHYS. RES. COMMUN. 40-45 (1993). The negative charge established by the intact mitochondrial membrane potential allows the lipophilic dye, bearing a delocalized positive charge, to enter the mitochondrial matrix where it accumulates. When the critical concentration is exceeded, J-aggregates form which become fluorescent red. Whereas, in apoptotic cells, the mitochondrial membrane potential collapses, and the JC-1 cannot accumulate within the mitochondria. In these cells JC-1 remains in the cytoplasm in a green fluorescent monomeric form. Apoptotic cells, showing primarily green fluorescence, are easily differentiated from healthy cells which show red and green fluorescence. The aggregate red form has absorption/emission maxima of 585/590 nm. Id. The green monomeric form has absorption/emission maxima of 510/527 nm. The JC-1 monomers and aggregates give strong positive signals, capable of yielding both qualitative and quantitative results. Detection methods include flow cytometry, fluorescence microscopy, spectrofluorometry, and a fluorescent 96-well plate reader format. Compounds and systems similar to JC-1, which stains mitochondria in a membrane potential-dependent fashion, can be utilized in the present invention.

Mitochondrial membrane potential can also be measured by non-fluorescent means, for example by using TTP (tetraphenylphosphonium ion) and a TTP-sensitive electrode (Porter and Brand, 269 AM. J. PHYSIOL. R1213 (1995); and Kamo et al., 49 J. MEMBRANE BIOL. 105 (1979)). Those skilled in the art will be able to select appropriate detectable compounds or other appropriate means for measuring MMP.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. See, e.g., Quinn, 1976 THE MOLECULAR BIOLOGY OF CELL MEMBRANES, University Park Press, Baltimore, Md., pp. 200-217.

In further embodiments, the present invention may measure other mitochondrial functions as a way of determining whether an organ is viable for transplantation. Such assays are known in the art and include, but are not limited to, reactive oxygen species (ROS) production, adenosine (ATP) generation, respiratory chain function, and mitochondrial protein cell sap protein ratios. The present invention may utilize one, some or all of these parameters to screen donor organs for use in transplantation.

In yet another aspect, the present invention provides kits for utilizing the methods described herein. The kits may comprise the equipment, solutions and instructions necessary to obtain a biopsy sample from a donor organ, isolate mitochondria from the sample, measure MMP and/or any other useful parameter in the isolated mitochondria. More specifically, a kit of the present invention may comprise needle biopsy kit components, mitochondria isolation kit components, and/or mitochondrial function assay kit components for measuring MMP, ROS production, ATP generation and/or mitochondrial respiratory chain function.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Measurement of Mitochondrial Function to Predict Transplant Outcome

Delayed graft function (DGF) is an important cause of morbidity and mortality among recipients of deceased donor renal transplants. Factors such as advanced donor age, prolonged cold ischemic time (CIT), and donation after cardiac death can portend poor graft function but none is predictive of DGF with great enough accuracy to influence pre-transplant graft selection. Mitochondrial function is widely accepted as an indicator of cell health and viability, and may represent a quantitative means to assess donor organ quality.

### Materials and Methods

Patients and Biopsies. Renal allograft needle biopsies performed from September 2007 to May 2008 in patients receiving a cadaveric renal transplant at The Johns Hopkins Transplant Center were studied. These included biopsies performed before reperfusion and at about thirty (30) to about sixty (60) minutes after reperfusion.

A total of twenty-six (26) renal transplants were performed during the study period specified above. For each biopsy, two-thirds (⅔) of the tissue was used for isolation of mitochondria. The remaining tissue was frozen in OCT (optimum cutting temperature) compound (Tissue-Tek; Sakura Finetek, Torrance, Cal.) at −20° C. The patient outcome and the level of MMP were double blind between surgeons and lab investigators until opening the codes. All study procedures were approved by the Institutional Review Board, Johns Hopkins Medical Institutions.

Mitochondrial Isolation. Biopsy samples were preserved in cold (4° C.) UW (University of Wisconsin) solution immediately after needle biopsy. Biopsy sample was transferred into 2 ml tube containing 1 ml cold extraction buffer (50 mM Tris-HCl). The tissue was homogenized using a tissue disruptor (Fisher Scientific, PowerGen 35) at high speed for 20 seconds, low speed for 10 seconds, followed by high speed for 10 seconds. The homogenates were centrifuged at 800 g (1,960 rpm) for 10 minutes at 4° C., and the supernatant was collected (the pellet containing nuclei was discarded). The supernatant was centrifuged at 8000 g (11,000 rpm) for 10 minutes at 4° C., and the pellet was re-suspended in 20 μl cold mitochondria storage buffer (Sigma, Cat. No. S9689). The mitochondria protein concentration was measured using the Bio-Rad Protein Assay Kit (Bio-Rad Laboratories, Inc., Hercules, Cal.).

Mitochondrial Membrane Potential Measurement. The JC-1 buffer was prepared as follows: 1 μl JC-1 Stain (5 mg) was added into 50 μl DMSO, mixed well, then added to 3.70 ml JC-1 assay buffer. The final concentration of JC-1 was 1.33 mg/ml.

Mitochondria were diluted to concentrations of 4 μg protein/25 μl with mitochondria storage buffer. 75 μl of JC-1 buffer was added to each well of a 96-well plate and the 25 μl of the mitochondrial suspension was added into each well. Each sample had three duplications.

After about 10 to 15 minutes at room temperature, relative fluorescence units (RFU) were detected at emission wavelengths 530 nm (monomer) to 590 nm (aggregates) by using a spectrofluorometer (FlexStation II) (Molecular Devices, Inc., Sunnyvale, Cal.). MMP was calculated as RFU/μg mitochondria.

### Results

Generally, mitochondria were isolated from pre- and post-reperfusion core needle biopsies obtained from 31 deceased-donor kidneys. Mitochondrial membrane potential (MMP) was measured via novel assay employing the fluorescent mitochondrial dye JC-1. MMP measurements were compared to graft cold ischemic time, donor age, and post-transplant recipient serum creatinine (Cr) levels.

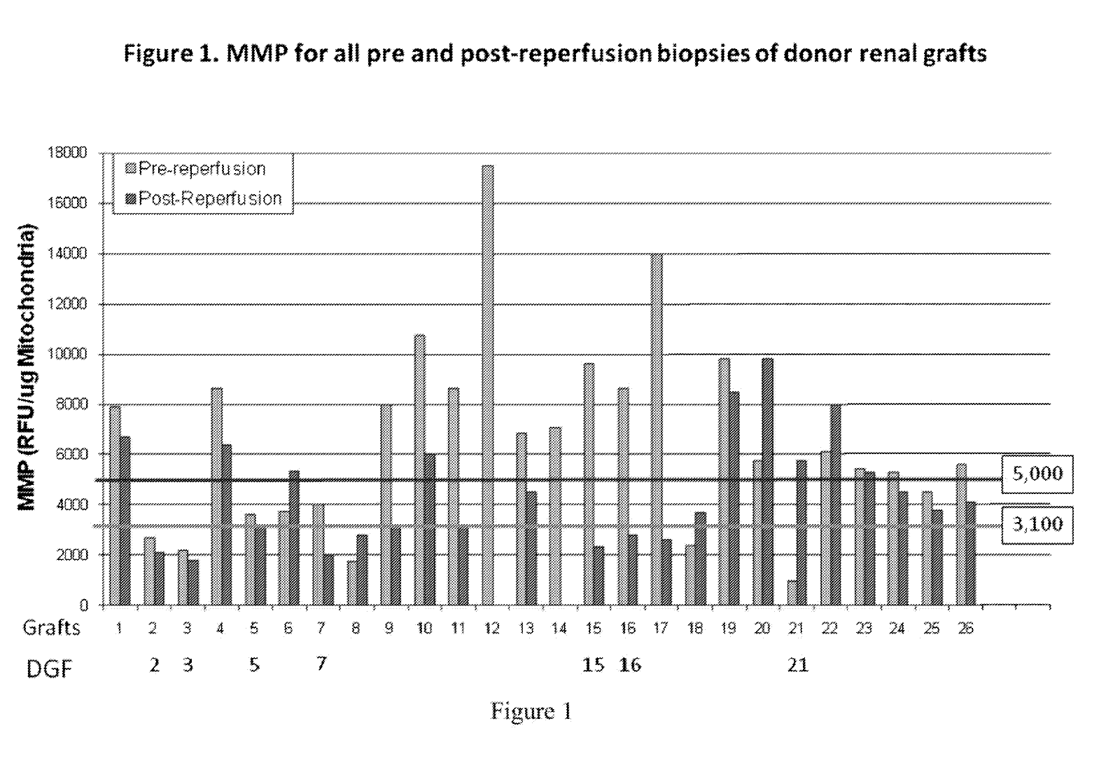

FIG. 1 shows the MMPs for all pre- and post-reperfusion biopsies of donor renal grafts. Delayed Graft Function (DGF) occurred in 9 out of 26 graft recipients (34.6%). In 8 biopsies, the MMP was less than 5000 (RFU/μg), and 87.5% (⅞) kidneys had DGF after transplantation. An additional two patients had DGF but had a “normal” MMP. Thus, the positive predictive value (normal function/normal MMP) was 88.8% ( 16/18), and the negative predictive value (DGF/poor MMP) was 87.5% (⅞).

In 10 biopsies, the MMP was less than 3100 (RFU/μg), and 70% ( 7/10) of these kidneys had DGF following transplantation. In 13 grafts, a pre-MMP less 5000 and/or post-MMP less 3100 were present and DGF occurred in 69% ( 9/13) of these kidneys following transplantation. Interestingly, all grafts (9/9) with DGF fit these categories.

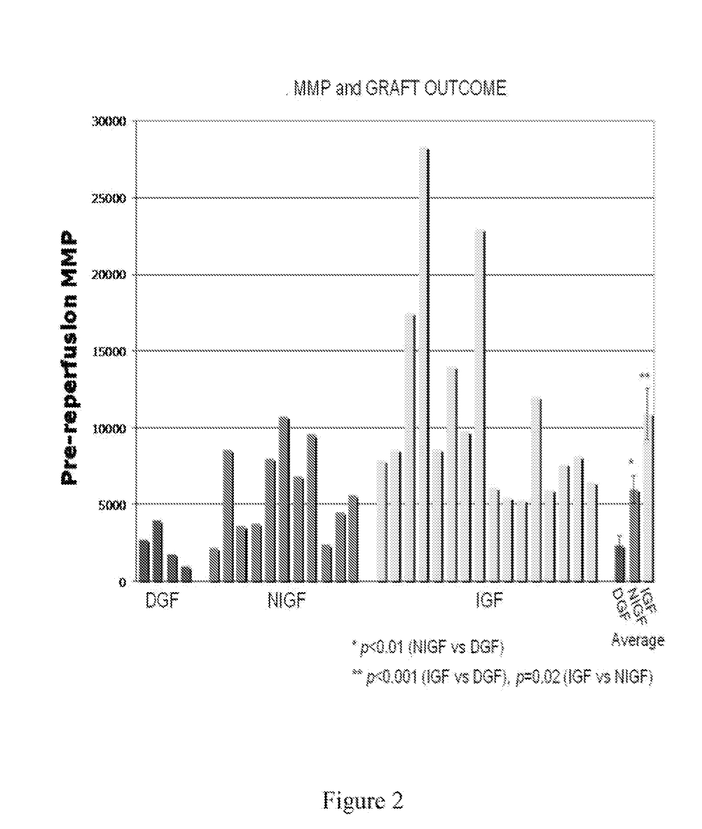

With reference to FIG. 2, recipients of biopsied grafts were classified as having Immediate Graft Function (IGF) if serum creatinine (Cr)≦3 mg/dl by post-operation day 5 (POD5), Non-Immediate (NIGF) (or Slow Graft Function (SGF)) if Cr>3 at POD5 but did not require dialysis, or Delayed Graft Function (DGF) if dialysis was required within one week of transplant. Mean MMP from DGF (n=4) grafts was 2353, while mean MMP from IGF (n=11) and NIGF (n=12) grafts was 11440 (p=0.01), and 7779 (p=0.01), respectively. 100% of IGF grafts had MMP>5000, while 100% of DGF grafts had MMP≦4000. A stepwise increase in MMP correlated with improved renal function.

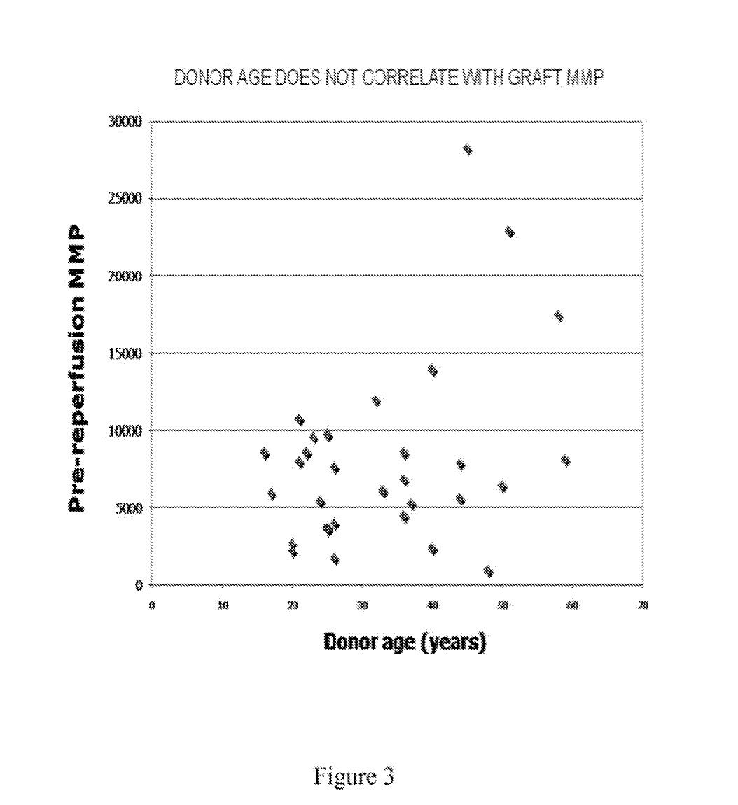

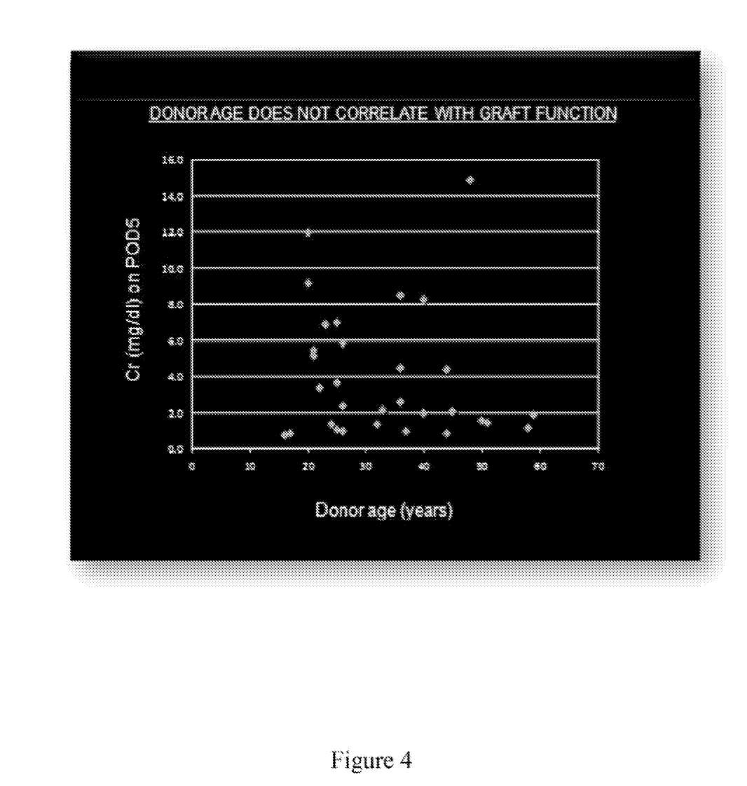

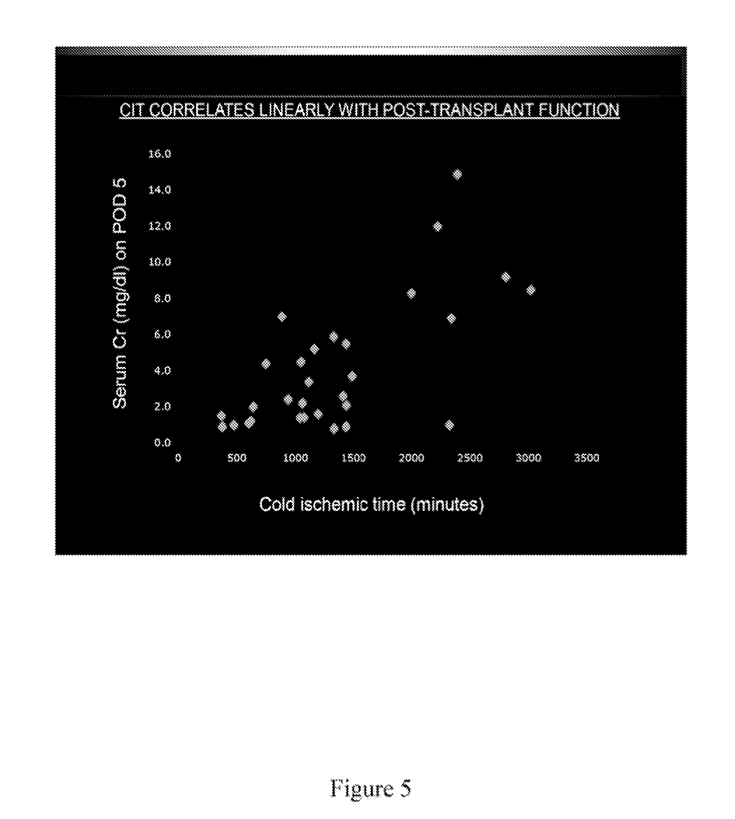

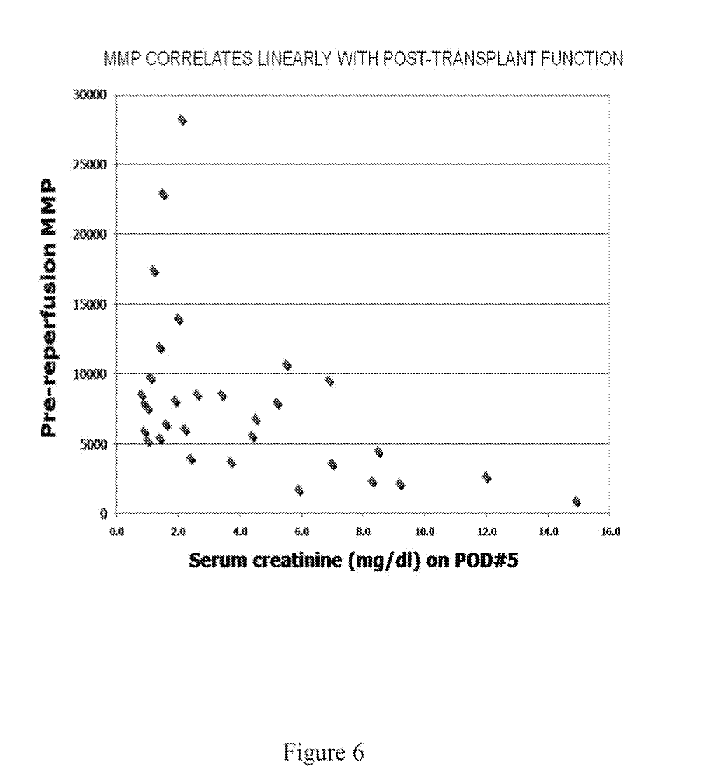

As shown in FIGS. 3 and 4, donor age does not correlate with graft MMP or graft function. The data in FIG. 5 shows that Cold Ischemic Time (CIT) correlates linearly with post-transplant function. In FIG. 6, MMP decreased in a linear fashion as cold ischemic times increased.

MMP can be reliably and quickly measured from needle biopsies taken from cold-preserved deceased donor renal grafts. Pre-reperfusion MMP correlates with post-transplant renal function, and may represent a novel and quantitative means by which to identify donor organs destined for DGF before these organs are engrafted. Screening the “marginal organs” by using MMP assays may increase the organ donor pool and decrease the risk of graft dysfunction after transplantation.

### Example 2

### Measurement of MMP in Liver Organ Transplantation

Mitochondria are isolated from pre- and post-reperfusion core needle biopsies obtained from a number of deceased-donor livers. MMP is measured using the assay described herein. MMP measurements are compared to graft cold ischemic time, donor age, and at least one post-transplant parameter indicative of graft function. All procedures are approved by the Johns Hopkins Hospital Institutional Review Board. It is expected that both pre- and post-reperfusion MMP correlates with CIT and with the post-transplant graft function. No correlation is expected between MMP and donor age.

### Example 3

### Measurement of MMP in Lung Transplantation

Mitochondria are isolated from pre- and post-reperfusion core needle biopsies obtained from a number deceased-donor lung. MMP is measured using the assay described herein. MMP measurements are compared to graft cold ischemic time, donor age, and at least one post-transplant parameter indicative of graft, function. All procedures are approved by the Johns Hopkins Hospital Institutional Review Board. It is expected that both pre- and post-reperfusion MMP correlates with CIT and with the post-transplant graft function. No correlation is expected between MMP and donor age.

### Example 4

### Measurement of MMP in Heart Transplantation

Mitochondria are isolated from pre- and post-reperfusion core needle biopsies obtained from a number deceased-donor hearts. MMP is measured using the assay described herein. MMP measurements are compared to graft cold ischemic time, donor age, and at least one post-transplant parameter indicative of graft function. All procedures are approved by the Johns Hopkins Hospital Institutional Review Board. It is expected that both pre- and post-reperfusion MMP correlates with CIT and with the post-transplant graft function. No correlation is expected between MMP and donor age.

### Example 5

### Measurement of Amp in Pancreas Transplantation

Mitochondria are isolated from pre- and post-reperfusion core needle biopsies obtained from a number of deceased-donor pancreases. MMP is measured using the assay described herein. MMP measurements are compared to graft cold ischemic time, donor age, and at least one post-transplant parameter indicative of graft function. All procedures are approved by the Johns Hopkins Hospital Institutional Review Board. It is expected that both pre- and post-reperfusion MMP correlates with CIT and with the post-transplant graft function. No correlation is expected between MMP and donor age.

### Example 6

### Measurement of MMP in Intestine Transplantation

Mitochondria are isolated from pre- and post-reperfusion core needle biopsies obtained from a number of deceased-donor intestines. MMP is measured using the assay described herein. MMP measurements are compared to graft cold ischemic time, donor age, and at least one post-transplant parameter indicative of graft function. All procedures are approved by the Johns Hopkins Hospital Institutional Review Board. It is expected that both pre- and post-reperfusion MMP correlates with CIT and with the post-transplant graft function. No correlation is expected between MMP and donor age.

### Example 7

### Additional Assays of Mitochondrial Function in Organ Transplantation

Additional assays of mitochondrial function are examined in organ transplants including, but not limited to, ROS production, respiratory chain function, and mitochondrial protein cell sap protein ratios. It is expected that such results correlate with CIT and with post-transplant graft function. No correlation is expected among such assay results and donor age.

### Example 8

### Measurement of MMP in Patients with Acute or Chronic Organ Dysfunction

Mitochondria are isolated from needle biopsies obtained from a number of patients with acute or chronic organ dysfunction due to ischemia, infections, drug injury or age. Organs can include kidney, liver, lung, heart, pancreas and/or intestine. MMP is measured using the assay described herein. MMP measurements are compared to diagnostic measurements of patient assessment, as well as patient outcome, as selected by one of ordinary skill in the art. All procedures are approved by the Johns Hopkins Hospital Institutional Review Board. It is expected that, in a statistically significant number of patients, MMP measurement correlates with patient outcome, more specifically, that MMP measurement is clinically useful in formulating prognosis in patients with acute or chronic organ dysfunction due to ischemia, infections, drug injury or age.

1. A method for predicting whether a donor organ is viable for transplantation comprising the steps of:
a. isolating mitochondria from a biopsy sample taken from a donor organ; and
b. measuring the mitochondrial membrane potential (MMP) of the isolated mitochondria,
wherein a MMP level that correlates to MMP levels from cells of a healthy organ indicates that the donor organ is a suitable candidate for transplantation.

2. The method of claim 1, wherein the organ is selected from the group consisting of kidney, liver, heart, pancreas, lung and intestine.

3. The method of claim 2, wherein the organ is a kidney.

4. The method of claim 3, wherein the MMP is at least 5000 RFU/μg mitochondria.

5. A method for predicting whether an organ will display immediate graft function following transplantation
a. isolating mitochondria from a biopsy sample taken from a donor organ; and
b. measuring the MMP of the isolated mitochondria,
wherein a MMP level that correlates to normal mitochondrial function in a healthy cell indicates that the donor organ is likely to exhibit immediate graft function following transplantation.

6. The method of claim 5, wherein the organ is selected from the group consisting of kidney, liver, heart, pancreas, lung and intestine.

7. The method of claim 6, wherein the organ is a kidney.

8. The method of claim 7, wherein the MMP is at least 5000 RFU/μg mitochondria.

9. A method for predicting organ transplant outcome comprising the step of measuring the MMP of mitochondria isolated from a biopsy taken from a donor organ, wherein a MMP level that correlates to altered mitochondrial function or cellular apoptosis is predictive of a negative organ transplant outcome.

10. A method for assessing the transplant viability of a donor kidney comprising the steps of:
a. isolating mitochondria from a biopsy sample taken from the donor kidney; and
b. measuring the MMP of the mitochondria,
wherein a MMP of at least 5000 RFU/μg mitochondria indicates that the donor organ is a viable candidate for transplantation.

11. A method for predicting whether a donor kidney will display immediate graft function following transplantation comprising the steps of:
a. isolating mitochondria from a biopsy sample taken from the donor kidney;
b. measuring the MMP of the mitochondria,
wherein a MMP of at least 5000 RFU/μg mitochondria is indicative that the donor kidney will display immediate graft function following transplantation.

12. The method of 11 wherein immediate graft function comprises a serum creatinine level of 3 mg/dl or less at post-operation day 5 (POD5).

13. A method for formulating a prognosis of patients with acute or chronic organ dysfunction comprising the steps of:
a. isolating mitochondria from a biopsy sample taken from the organ; and
b. measuring the MMP of the isolated mitochondria,
wherein a MMP level that correlates to normal mitochondrial function in a healthy organ is predictive of a positive prognosis, and wherein a MMP level that correlates to altered mitochondrial function or cellular apoptosis is predictive of a negative prognosis.

14. The method of claim 13, wherein the organ dysfunction results from ischemia, infection, drug injury, or age.

15. A kit useful for assessing donor organ viability for transplantation comprising:
a. needle biopsy kit components; and
b. mitochondria isolation kit components.

16. The kit of claim 15, further comprising mitochondrial function assay kit components for measuring reactive oxygen species production, adenosine triphosphate generation, respiratory chain function, and/or mitochondrial protein cell sap protein ratios.
